(19) United States
(12) Patent Application Publication
(10) Pub. No.: US 2023/0114656 A1
(43) Pub. Date: Apr. 13, 2023

(54) PRODUCTS AND METHODS FOR EXTENDING THE SHELF LIFE OF WATER SENSITIVE PRODUCTS

(71) Applicant: JDS Therapeutics, LLC, Harrison, NY (US)

(72) Inventors: James R. Komorowski, Trumbull, CT (US); Haley Komorowski, Trumbull, CT (US)

(21) Appl. No.: 17/958,303

(22) Filed: Sep. 30, 2022

Related U.S. Application Data
(60) Provisional application No. 63/253,920, filed on Oct. 8, 2021.

(51) Int. Cl. A61K 47/42; A61K 35/741; A61K 47/24; A23L 33/135; A23L 33/17

(52) U.S. Cl. CPC A61K 47/42; A61K 35/741; A61K 47/24; A23L 33/135; A23L 33/17; A61K 2035/115

(57) ABSTRACT

Compositions comprising lactoferrin are described herein, which can allow for the use of ingredients in a formulation that previously could not be used, or combined together without encountering stability problems. The lactoferrin can also be included so as to eliminate the need for capsule-in-capsule formulations or embodiments comprising lactoferrin and a probiotic. As described herein, certain embodiments of the disclosure can comprise an active ingredient. An active ingredient can comprise one or more of a probiotic formulation, nutritional supplement, and a foodstuff. In certain embodiments, a probiotic formulation, nutritional supplement, and/or foodstuff. In certain embodiments, an active ingredient is a high water activity ingredient.

### CROSS-REFERENCE TO RELATED APPLICATION

This application claims priority to U.S. Provisional Application No. 63/253,920 filed on Oct. 8, 2021, the disclosure of which is incorporated herein by reference in its entirety.

### BACKGROUND

Probiotic products, other water-sensitive formulations, and foods, have a limited shelf life due to their sensitivity to water, making them susceptible to negative interactions with other ingredients included in the formulation(s). This susceptibility can limit the ingredients and types of ingredients that can be successfully added to or combined in various compositions. Existing technologies to solve the compatibility problem include the use of physical barriers, such as compartmentalizing components, or utilizing more complex arrangements such as a capsule-in-a-capsule technology to separate incompatible ingredients from water-sensitive products, such as probiotics. Another existing technology is that probiotic spores are used, which provides the probiotic with a protective coating to prevent interactions with incompatible ingredients, but these options have not adequately solved the problems.

The present invention provides lactoferrin compositions and methods of incorporating lactoferrin into compositions to solve problems associated with hydration of products over time. The present invention also allows for a wider variety of probiotic strains to be used without the need for the probiotic to be a spore or the probiotic formulation to be separated from the other ingredients.

### DETAILED DESCRIPTION

Certain embodiments comprise lactoferrin, which can allow for the use of ingredients in a formulation that previously could not be used, or combined together without encountering stability problems. Lactoferrin can also be included so as to eliminate the need for capsule-in-capsule formulations or embodiments comprising lactoferrin and a probiotic.

As described herein, certain embodiments of the disclosure can comprise an active ingredient. An active ingredient can comprise one or more of a probiotic formulation, nutritional supplement, and a foodstuff. In certain embodiments, an active ingredient is a high water activity ingredient.

Certain embodiments can comprise lactoferrin and a high water activity ingredient. Without being bound by any particular theory, lactoferrin (a globular glycoprotein from the transferrin family) can be combined with one or more high water activity ingredients, thereby increasing the compositions shelf life, bacterial growth inhibition, and freshness. The lactoferrin can also reduce clumping in the composition, stabilize the composition's water activity, and/or reduce the composition's water activity.

A nutritional supplement can be chosen from the group consisting of phycocyanin peptides, phycocyanin oligopeptides, arginine, L-arginine, L-citrulline, rose hips extract, flower pollen extract, pistil extract, royal jelly, magnesium picolinate, chromium, zinc gluconate, zinc picolinate, maca powder, maca extracts, spirulina extract, fungal extracts such as one or more of Cordyceps Sinensis, Reishi (Ganoderma lucidum), Lion's Mane (Hericium erinaceus), and King trumpet (Pleurotus eryngii), L-carnitine, creatine, one or more amino acids, vitamin D, thiamin, niacin, vitamin B6, pantothenic acid, calcium, magnesium, sodium, potassium, nootropic agents, Rhodiola crenulata, rhizome extract, caffeine anhydrous, alpha-yohimbine, yohimbine, ginseng, melatonin, antioxidants, potassium, selenium, coenzyme Q10, pine bark extract, pterostilbene, nicotinamide riboside, beta-carotene, lycopene, lutein, zeaxanthin, grape seed extract, curcumin, biotin (vitamin B7, vitamin H), choline (vitamin Bp), folate (folic acid, vitamin Bp, vitamin M), niacin (vitamin B3, vitamin P, vitamin PP), pantothenic acid (vitamin B5), riboflavin (vitamin B2, vitamin G), thiamine (vitamin B1), vitamin A (retinol), vitamin B6 (pyridoxine, pyridoxamine, or pyridoxal), vitamin B12 (cobalamin), vitamin C (ascorbic acid), vitamin E (tocopherol), and vitamin K (naphthoquinoids) and the like.

Certain embodiments described herein can comprise a probiotic formulation. A probiotic formulation can consist of one or more of Lactobacillus acidophilus, Lactobacillus rhamnosus, Bacillus clausii, Lactobacillus casei, Bifidobacterium bifidum, Lactobacillus paracasei, such as Lpc-37, Bifidobacterium lactis, Streptococcus thermophiles, Bifidobacterium breve, Bifidobacterium longum, Bifidobacterium infantis, Lactobacillus casei Shirota, and the like.

Certain embodiments can comprise a food wherein the food comprises one or more high water activity foodstuff. A foodstuff can comprise one or more of raw vegetables, raw fruits, cooked vegetables, cooked fruits, meat, peppers, broccoli, oranges, apples, chicken, beef, bread, jellies, jams, gummy items, snack food such as potato chips, pretzels, tortilla chips, nuts, bread, cake, confectionaries, pastries, grains, food powders such as cocoa powder, peanut butter powder, milk powder, dried fruits, beef jerky, flour, spices, cured meats such as salami, pepperoni, ham, and the like, cheese, ground coffee, food pastes such as bean paste, curry paste, tomato paste, and the like. In some embodiments, a food comprises one or more foodstuffs and the food is frozen. In some embodiments, a food and/or foodstuff can be freeze-dried. In some embodiments, a food is a survival food, e.g., food utilized for camping, long-term storage for emergencies, or any other food intended to be stored for more than 6, 12, 18 or 24 months or longer.

Certain embodiments may further comprise one or more excipients. An excipient can comprise one or more of droxypropylmethylcellulose, methyl cellulose, polyvinylpyrollidone, citric acid, binders, fillers, starch, sucrose and/or other sugars, polyethylene glycol, and any natural gum such as acacia tragacanth, alginates, chitosan, xanthan, pectin, and other excipients known in the art. Certain embodiments can further comprise preservatives such as a natural preservative.

In some embodiments, the lactoferrin may be present in 0-1%, 0-5%, 0-10%, 5-10%, 10-15%, 10-25%, 5-30%, 25-50%, or 25-75% by weight of the composition, and any value therebetween.

Compositions described herein can be used to increase the shelf life of a food, increase the shelf life of a nutritional supplement, increase the shelf life of a probiotic formulation, and combinations thereof. In some embodiments, the compositions described herein can be used to increase the stability of a high water activity ingredient and compositions comprising one or more high water activity ingredients. Compositions described herein can be used to increase the stability of a food, nutritional supplement, and/or probiotic formulation. In certain embodiments, compositions described herein are applied to a food to prevent spoilage.

Certain embodiments described herein comprise a method of manufacturing a composition wherein the composition comprises one or more of a food, nutritional supplement, and/or a probiotic formulation. Certain embodiments described herein can be used to inhibit bacterial growth in a composition wherein the composition comprises a food, nutritional supplement, and/or probiotic formulation. Certain compositions described herein can be used to reduce the likelihood of food poisoning, by reducing the rate of bacterial growth in the composition. In certain embodiments, lactoferrin is included in a composition to reduce the rate of spoilage, reduce the growth of bacteria, inhibit the rate of bacterial growth, increase the stability, increase the shelf life, preserve, maintain the activity of an active ingredient, and a combination thereof of the composition.

A container-type that can house a composition of the present disclosure is not particularly limited. In some embodiments, the compositions disclosed herein are stored in bottles, pouches, bags, sealed containers, jars, cans, and the like. Some embodiments provide solid dosage forms of the compositions disclosed herein. Embodiments described herein comprising compositions disclosed herein as a nutritional supplement means that the composition disclosed herein is present in an unnatural form, i.e., is presented in a supplement (e.g., in a pill or powder) that is different from that which occurs naturally, or the nutritional or dietary supplement results in unnatural supplementation that is unachievable through a non-supplemented diet.

For oral administration, the compositions disclosed herein can be provided as a tablet, dispersible powder or granule, emulsion, hard or soft capsule, syrup, or elixir. Solid dosage forms such as tablets and capsules may comprise an enteric coating. Compositions intended for oral use can be prepared according to any method known in the art for the manufacture of pharmaceutically acceptable compositions and such compositions may include one or more of the following agents: sweeteners, flavoring agents, coloring agents, coatings, and preservatives. The sweetening and flavoring agents will increase the palatability of the preparation. Tablets containing the complexes in admixture with non-toxic pharmaceutically acceptable excipients suitable for tablet manufacture are acceptable. Pharmaceutically acceptable vehicles such as excipients are compatible with the other ingredients of the formulation (as well as non-injurious to the patient). Such excipients include inert diluents such as calcium carbonate, sodium carbonate, lactose, calcium phosphate or sodium phosphate; granulating and disintegrating agents, such as corn starch or alginic acid; binding agents such as starch, gelatin, or acacia; and lubricating agents such as magnesium stearate, stearic acid, or talc. Tablets can be uncoated or can be coated by known techniques to delay disintegration and absorption in the gastrointestinal tract and thereby provide a sustained action over a longer period of time. For example, a time delay material such as glyceryl monostearate or glyceryl distearate alone or with a wax can be employed.

Formulations for oral use can also be presented as hard gelatin-containing or non-gelatinous capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin, or olive oil. In certain embodiments the dosage form is not a capsule-in-capsule formulation.

As used in the claims below and throughout this disclosure, the phrase “consisting essentially of” is meant to include any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and can or cannot be present depending upon whether or not they affect the activity or action of the listed elements. For example, the use of a composition “consisting essentially of a composition” for the treatment of a particular disease or disorder, or the maintenance of a healthy condition, would exclude other ingredients that would materially alter the intended outcome of the composition.

As used herein, a composition that “substantially” comprises a compound means that the composition contains more than about 80% by weight, more preferably more than about 90% by weight, even more preferably more than about 95% by weight, and most preferably more than about 98% by weight of the compound.

The term “pharmaceutical formulation”, “formulation”, “composition” and the like can refer to preparations which are in such a form as to permit the biological activity of the active ingredients to be effective, and therefore may be administered to a subject for therapeutic use along with dietary and/or nutritional supplement use. The meaning of these terms will be clear to the skilled artisan based upon the context in which they are used.

A “therapeutically effective amount” as used herein includes within its meaning a non-toxic but sufficient amount of a compound active ingredient or composition comprising the same for use in the embodiments disclosed herein to provide the desired therapeutic effect. Similarly “an amount effective to” or “an effective amount” as used herein includes within its meaning a non-toxic but sufficient amount of a compound active ingredient or composition comprising the same to provide the desired effect. A “therapeutically effective amount” or an “effective amount” includes amounts of compounds that would not be achievable through a standard diet, but requires supplementation and dosing as described herein. The exact amount of the active ingredient disclosed herein required will vary from subject to subject depending on factors such as the species being treated, the age and general condition of the subject, the severity of the condition being treated, the particular agent being administered, the weight of the subject, and the mode of administration and so forth. Thus, it may not always be possible to specify an exact “effective amount.” However, for any given case, an appropriate “effective amount” may be determined by one of ordinary skill in the art in view of the disclosure contained herein. In some aspects, a therapeutically effective amount may include a dosing regimen. For example, a therapeutically effective amount may include about 100 mg of a composition orally consumed each day for fourteen consecutive days. In some aspects, a therapeutically effective amount may include about 100 mg of a composition orally consumed each day for thirty consecutive days. Compositions including a composition may include, for example, between 0.1-1000 grams of the composition.

Water activity (A_{w}) is a measurement of unbound water in a sample. Water that is not bound to an ingredient can be used by unwanted microorganisms, which could lead to one of the contributing factors for food spoilage. Water activity is a thermodynamic measure of water expressed as the vapor pressure of water in a sample divided by vapor pressure of pure water at a given temperature:

A_{w}=P/P₀

Where P refers to the vapor pressure in a food or ingredient and P₀ is the vapor pressure of pure water.

Water Activity is based on a scale from 0.0 to 1.0. A water activity greater than 0.2 A_{w} is considered not acceptable for probiotic formulations since the higher level of unbound water can stimulate growth of a previously freeze-dried bacteria. The probiotic bacteria in a capsule, for instance, will then die without a source of food, rendering the product not effective when consumed.

As used herein, the term “high water activity” generally refers to a composition with a water activity greater than about 0.2 A_{w}. Higher water activity compositions can comprise probiotics.

A variety of ingredients were tested for water activity (See Table 1). The majority of ingredients had a water activity above 0.2 A_{w}. These ingredients would not be useable in a probiotic capsule formulation. A few ingredients were below 0.2 A_{w}, and these would be suitable ingredients to combine with probiotics in a capsule.

TABLE 1
Water Activity of Ingredients

| | Water Activity | Temp. |
|---|---|---|
| Name | (A_{w}) | (° Celsius) |
| MLF 2M Lactoferrin | 0.0456 | 24.94 |
| WCB Lactoferrin | 0.0511 | 24.96 |
| Magnesium Biotinate | 0.1075 | 24.98 |
| Phycocyanin Peptides | 0.1986 | 24.99 |
| Arginine Silicate Inositol (ASI) PASD | 0.208 | 25.03 |
| Arginine Silicate Inositol and Free Inositol | 0.2392 | 25.04 |
| GC-FEM Flower Pollen Ex | 0.3115 | 25.04 |
| PI-82 Flower Pollen Ex | 0.3275 | 25.03 |
| Magnesium Picolinate Dihydrate | 0.3391 | 25.03 |
| Magnesium L-Theanate | 0.3408 | 25.06 |
| Zinc Gluconate | 0.3522 | 25.04 |
| Zinc Picolinate | 0.3528 | 25.1 |
| Maca | 0.3813 | 24.98 |
| Spirulina Supplement | 0.3834 | 25.04 |
| Chromium Picolinate | 0.3834 | 25.05 |
| Magnesium L-Theanate + NaCl | 0.4056 | 25.05 |

The impact of mixing one ingredient with a low water activity with another ingredient that has a high water activity was evaluated. The goal is that the ingredient with the lower water activity reduces the impact of the ingredient with the higher water activity when mixed together. The tests were performed to determine the overall water activity of a 50:50 mixture of the two ingredients, with the prediction that the resulting water activity would be the average water activity (i.e., a 50:50 mixture of an ingredient with a 0.2 A_{w} and an ingredient with a 0.4 A_{w} would result in a combined water activity of 0.3 A_{w}).

### EXAMPLES

### Example 1 (Table 2)

A combination of ASI (high water activity component) and magnesium biotinate (low water activity component) was tested. The ASI sample had a water activity of 0.2080 A_{w} and the magnesium biotinate sample had a water activity of 0.1075 A_{w}. A 50:50 combination was predicted to have a water activity of 0.1578 A_{w}. (based on average water activity calculation). The results showed that the combination sample had a water activity of 0.1489 A_{w}. These results are very close to each other, confirming the expected result.

TABLE 2
Combination of ASI and Magnesium Biotinate (50:50)
with an expected A_{w} of 0.1578. Testing resulted in
a similar A_{w} reading of 0.1489.

| ASI (50%) + Magnesium Biotinate (50%) | | | |
|---|---|---|---|
| | Water Activity | Temp. | |
| Weight (mg) | (A_{w)} | (Celsius) | |
| 1000 | 0.1213 | 24.98 | |
| 1010 | 0.1475 | 25.05 | Average Water Activity: |
| | | | 0.1489 A_{w} |
| 870 | 0.1779 | 25.03 | |

### Example 2 (Table 3)

A combination of zinc picolinate (high water activity component) and lactoferrin (low water activity component) was tested. The lactoferrin sample had a water activity of 0.0456 A_{w}, the zinc picolinate sample had a water activity of 0.3528 A_{w}. Based on the results above, a 50:50 combination of lactoferrin and zinc picolinate was predicted to have a water activity of 0.1992 A_{w}. However, the results show that this combination had an average water activity of 0.1160 A_{w}. Significantly lower than the expected value.

TABLE 3
Combination of lactoferrin and zinc picolinate (50:50)
with an expected A_{w} of 0.1992. Testing resulted in an
unexpectedly low A_{w} reading of 0.1160.

| Lactoferrin (50%) + Zinc Picolinate (50%) | | | |
|---|---|---|---|
| | Water Activity | Temp. | |
| Weight (mg) | (A_{w)} | (Celsius) | |
| 1000 | 0.0741 | 24.97 | |
| 1000 | 0.1111 | 24.98 | Average Water Activity: |
| | | | 0.1160 A_{w} |
| 1000 | 0.163 | 24.99 | |

### Example 3 (Table 4)

A combination of zinc gluconate (high water activity component) and lactoferrin (low water activity component) was tested. The lactoferrin sample had a water activity of 0.0456 A_{w}, and the zinc gluconate sample had a water activity of 0.3522 A_{w}. Based on these results, a 50:50 combination of lactoferrin and zinc picolinate was predicted to have a water activity of 0.1989 A_{w}. However, the results show that this combination had an average water activity of 0.1382 A_{w}. Significantly lower than the expected value.

TABLE 4
Combination of lactoferrin and zinc gluconate (50:50)
with an expected A_{w} of 0.1989. Testing resulted in an unexpectedly
low A_{w} reading of 0.1382.

| Lactoferrin (50%) + Zinc Gluconate (50%) | | | |
|---|---|---|---|
| | Water Activity | Temp. | |
| Weight (mg) | (A_{w)} | (Celsius) | |
| 990 | 0.0994 | 24.94 | |
| 1010 | 0.1271 | 24.98 | Average Water Activity: |
| | | | 0.1382 A_{w} |
| 1000 | 0.1882 | 24.99 | |

As will be appreciated by those skilled in the art, the uses and methods provided herein may be applicable to multiple formulations that require adequate protection against unwanted interactions with unbound water as it relates to water activity within a composition.

In certain embodiments of the invention, various combinations of probiotics and/or other high water activity ingredients in the compositions are formulated together, wherein the formulation presents improved protection against the high sensitivity of the bacteria being highly susceptible to negatively interacting with other ingredients included in the formulation. These formulations utilize lactoferrin in necessary quantities to inhibit unwanted interactions of the ingredients to unbound water within the compositions and formulations. In certain embodiments, the quantities of lactoferrin present in the compositions and formulations controls and/or reduces the water activity.

In certain embodiments, the probiotics can be individually formulated or in any particular combination of probiotics and/or one or more high water activity ingredients, for inclusion with the lactoferrin-containing mixes of the invention. Some embodiments provide probiotic compositions in various formulations including, for example, capsules, soft gel caps, hard gel caps, powders, tablets, granules, microencapsulations, and the like.

### Example 4

A study will be conducted where lactoferrin is added at different amounts (wt. %), to a food or supplement that has a high water activity, thereby having a shortened shelf life compared to lower water activity ingredients. A series of foodstuffs and/or nutritional supplements will be analyzed to ascertain their baseline activity (e.g., NK3 antagonism will be measured for an NK3 antagonist). A foodstuff or supplement will then be combined with lactoferrin and subjected to an accelerated stability test where the temperature of the composition is raised and the humidity of the environment in which the composition is stored, is raised as well (e.g., 40° C.±2° C./75% RH±5% RH for 3 to 6 months). Different temperatures and humidities will be utilized to assay the results of different combinations of lactoferrin and foodstuffs and/or nutritional supplements. Other conditions may also be altered as appropriate for the foodstuff/supplement testing. The activities of the foodstuff and/or nutritional supplements will be re-tested to ascertain the difference in activity levels in the composition between the initial activity and activity after the stability test to see if they degrade less or not at all when the lactoferrin was added, compared to the Control (no lactoferrin added). Other “non-active” parameters will be measured and compared to initial values, such as hardness and color and other visual changes that can occur with declining stability.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

What is claimed is:

1. A method of increasing the shelf life of a probiotic formulation, the method comprising adding lactoferrin to the probiotic formulation, wherein the probiotic formulation comprises at least one probiotic strain and at least one high water activity ingredient.

2. The method of claim 1, wherein the at least one high water activity ingredient is selected from the group consisting of phycocyanin peptides, phycocyanin oligopeptides, arginine, L-arginine, L-citrulline, rose hips extract, flower pollen extract, pistil extract, royal jelly, magnesium picolinate, chromium, zinc gluconate, zinc picolinate, maca powder, maca extracts, spirulina extract, fungal extracts such as one or more of Cordyceps Sinensis, Reishi (Ganoderma lucidum), Lion's Mane (Hericium erinaceus), and King trumpet (Pleurotus eryngii), L-carnitine, creatine, one or more amino acids, vitamin D, thiamin, niacin, vitamin B6, pantothenic acid, calcium, magnesium, sodium, potassium, nootropic agents, Rhodiola crenulata, rhizome extract, caffeine anhydrous, alpha-yohimbine, yohimbine, ginseng, melatonin, antioxidants, potassium, selenium, coenzyme Q10, pine bark extract, pterostilbene, nicotinamide riboside, beta-carotene, lycopene, lutein, zeaxanthin, grape seed extract, curcumin, biotin (vitamin B7, vitamin H), choline (vitamin Bp), folate (folic acid, vitamin Bp, vitamin M), niacin (vitamin B3 vitamin P, vitamin PP), pantothenic acid (vitamin B5), riboflavin (vitamin B2, vitamin G), thiamine (vitamin B1), vitamin A (retinol), vitamin B6 (pyridoxine, pyridoxamine, or pyridoxal), vitamin B12 (cobalamin), vitamin C (ascorbic acid), vitamin E (tocopherol), and vitamin K (naphthoquinoids), and combinations thereof.

3. The method of claim 1, wherein the probiotic formulation is formulated as one selected from the group consisting of a capsule, a soft gel cap, a hard gel cap, a powder, a tablet, a granule, and a microencapsulation.

4. The method of claim 1, wherein the at least one or more probiotic strains are not formulated as probiotic spores.

5. The method of claim 1, wherein the lactoferrin is at most 75% by weight of the probiotic formulation.

6. A probiotic formulation, the probiotic formulation comprising:
at least one probiotic strain;
at least one high water activity ingredient; and
lactoferrin.

7. The probiotic formulation of claim 6, wherein the at least one high water activity ingredient is selected from the group consisting of phycocyanin peptides, phycocyanin oligopeptides, arginine, L-arginine, L-citrulline, rose hips extract, flower pollen extract, pistil extract, royal jelly, magnesium picolinate, chromium, zinc gluconate, zinc picolinate, maca powder, maca extracts, spirulina extract, fungal extracts such as one or more of Cordyceps Sinensis, Reishi (Ganoderma lucidum), Lion's Mane (Hericium erinaceus), and King trumpet (Pleurotus eryngii), L-carnitine, creatine, one or more amino acids, vitamin D, thiamin, niacin, vitamin B6, pantothenic acid, calcium, magnesium, sodium, potassium, nootropic agents, Rhodiola crenulata, rhizome extract, caffeine anhydrous, alpha-yohimbine, yohimbine, ginseng, melatonin, antioxidants, potassium, selenium, coenzyme Q10, pine bark extract, pterostilbene, nicotinamide riboside, beta-carotene, lycopene, lutein, zeaxanthin, grape seed extract, curcumin, biotin (vitamin B7, vitamin H), choline (vitamin Bp), folate (folic acid, vitamin Bp, vitamin M), niacin (vitamin B3, vitamin P, vitamin PP), pantothenic acid (vitamin B5), riboflavin (vitamin B2, vitamin G), thiamine (vitamin B1), vitamin A (retinol), vitamin B6 (pyridoxine, pyridoxamine, or pyridoxal), vitamin B12 (cobalamin), vitamin C (ascorbic acid), vitamin E (tocopherol), and vitamin K (naphthoquinoids), and combinations thereof.

8. The probiotic formulation of claim 6, wherein the probiotic formulation is formulated as one selected from the group consisting of a capsule, a soft gel cap, a hard gel cap, a powder, a tablet, a granule, and a microencapsulation.

9. The probiotic formulation of claim 6, wherein the at least one or more probiotic strains are not formulated as probiotic spores.

10. The probiotic formulation of claim 6, wherein the lactoferrin is at most 75% by weight of the probiotic formulation.

11. A nutritional supplement, the nutritional supplement comprising:
at least one active ingredient;
at least one high water activity ingredient; and
lactoferrin.

12. The nutritional supplement of claim 11, wherein the at least one high water activity ingredient is selected from the group consisting of phycocyanin peptides, phycocyanin oligopeptides, arginine, L-arginine, L-citrulline, rose hips extract, flower pollen extract, pistil extract, royal jelly, magnesium picolinate, chromium, zinc gluconate, zinc picolinate, maca powder, maca extracts, spirulina extract, fungal extracts such as one or more of Cordyceps Sinensis, Reishi (Ganoderma lucidum), Lion's Mane (Hericium erinaceus), and King trumpet (Pleurotus eryngii), L-carnitine, creatine, one or more amino acids, vitamin D, thiamin, niacin, vitamin B6, pantothenic acid, calcium, magnesium, sodium, potassium, nootropic agents, Rhodiola crenulata, rhizome extract, caffeine anhydrous, alpha-yohimbine, yohimbine, ginseng, melatonin, antioxidants, potassium, selenium, coenzyme QM, pine bark extract, pterostilbene, nicotinamide riboside, beta-carotene, lycopene, lutein, zeaxanthin, grape seed extract, curcumin, biotin (vitamin B7, vitamin H), choline (vitamin Bp), folate (folic acid, vitamin Bp, vitamin M), niacin (vitamin B3, vitamin P, vitamin PP), pantothenic acid (vitamin B5), riboflavin (vitamin B2, vitamin G), thiamine (vitamin B1), vitamin A (retinol), vitamin B6 (pyridoxine, pyridoxamine, or pyridoxal), vitamin B12 (cobalamin), vitamin C (ascorbic acid), vitamin E (tocopherol), and vitamin K (naphthoquinoids), and any combination thereof.

13. The nutritional supplement of claim 11, wherein the active ingredient is a probiotic formulation.

14. The nutritional supplement of claim 11, wherein the at least one or more probiotic strains are not formulated as probiotic spores.

15. The probiotic formulation of claim 11, wherein the lactoferrin is at most 75% by weight of the probiotic formulation.

16. A food comprising:
a foodstuff with high water activity;
lactoferrin.

17. The food of claim 16, wherein the foodstuff is selected from the group consisting of raw vegetables, raw fruits, cooked vegetables, cooked fruits, meat, peppers, broccoli, oranges, apples, chicken, beef, bread, jellies, jams, gummy items, snack food such as potato chips, pretzels, tortilla chips, nuts, bread, cake, confectionaries, pastries, grains, food powders such as cocoa powder, peanut butter powder, milk powder, dried fruits, beef jerky, flour, spices, cured meats such as salami, pepperoni, ham, and the like, cheese, food pastes such as bean paste, curry paste, tomato paste, and combinations thereof.

18. A method of increasing the shelf life of a composition, wherein the composition comprises lactoferrin and a high water activity ingredient.

19. A method of inhibiting bacterial growth in a composition, wherein the composition comprises lactoferrin and a high water activity ingredient.

20. A method of increasing the stability of a composition, wherein the composition comprises lactoferrin and a high water activity ingredient.
